(19) United States
(12) Patent Application Publication
(10) Pub. No.: US 2023/0405385 A1
(43) Pub. Date: Dec. 21, 2023

(54) THERAPEUTIC OR EXERCISE ELASTOMER PUTTY SPECIALLY FORMULATED WITH CBD OIL AND/OR DMSO TO REDUCE PAIN AND INFLAMMATION DURING HAND EXERCISE OR REHABILITATION

(71) Applicant: DEPCO, INC., Hauppauge, NY (US)

(72) Inventors: M. Gregory Minuto, Smithtown, NY (US); Stephen Mlcoch, Manorville, NY (US)

(21) Appl. No.: 18/200,925

(22) Filed: May 23, 2023

Related U.S. Application Data
(60) Provisional application No. 63/345,097, filed on May 24, 2022.

(51) Int. Cl. A63B 21/02; A61K 31/00; A61K 47/20; A61K 47/34

(52) U.S. Cl. CPC A63B 21/028; A61K 31/658; A63B 23/16; A61K 47/34; A61K 47/20

(57) ABSTRACT

A therapeutic or exercise putty includes a boronated silicone elastomer serving as the base ingredient or component and CBD or CBG oil or powder, DMSO, or CBD or CBG oil or powder together with DMSO, as a secondary ingredient mixed with the base putty. The inclusion of any one of the aforementioned secondary ingredients will reduce the pain and inflammation when the putty is used for hand rehabilitation or exercise by users and patients. Added side effects of reduced stress, tension or discomfort also contribute to a positive rehabilitation exercise experience that supports better user or patient health.

### CROSS-REFERENCE TO RELATED APPLICATIONS

This application is related to U.S. Provisional Application Ser. No. 63/345,097, filed on May 24, 2022, and titled “Therapeutic Or Exercise Elastomer Putty Specially Formulated With CBD Oil And/Or DMSO To Reduce Pain And Inflammation During Hand Exercise Or Rehabilitation”, the disclosure of which is incorporated herein by reference and on which priority is hereby claimed.

### BACKGROUND OF THE INVENTION

### Field of the Invention

The present invention relates to therapeutic exercise putties specially made for use by the public and patients seeking hand exercise methods or patients who have strength issues or require rehabilitation.

### Description of the Related Art

Many orthopedic and physical therapists treat patients who have hand maladies. These maladies cover a wide range of issues from people who have strength issues to those who have issues with arthritis or recovery needs from an injury. Much of the rehabilitation is done through exercise. One of the ways exercise is done is with therapeutic putty. This method offers strength building and rehabilitation options for the hand and even circulation issues in the arm.

One issue, which is part of hand malady problems, is the association of pain or inflammation involved. During rehabilitation activities, this concern is part of the need to exercise or rehabilitate the hand, especially for people that are not perfectly healthy or that are recovering and need to sustain hand exercise as long as is possible or needed according to a therapist or health care provider. Reducing pain and inflammation during rehabilitation and exercise activities is an essential objective in treating patients in order to attain good health. Methods that permit patients and the public to rehabilitate without the agony, stress or suffering that could be involved with such activity, offers an improved treatment.

### OBJECTS AND SUMMARY OF THE INVENTION

It is an object of the present invention to provide a therapeutic or exercise putty, which helps resolve the problem and concern of pain or inflammation, during rehabilitation and exercise using a hand exercise putty.

The invention is directed to a therapeutic or exercise putty, which is manipulated within the hand in different ways by the user and which includes a base putty formulation, such as boronated silicone elastomer and a CBD (cannabidiol) or CBG (cannabigerol) oil or powder and/or DMSO (dimethysulfoxide) such as disclosed herein or equivalents thereof added to and mixed with the base putty formulation that will act to alleviate pain or inflammation when a user or patient is undergoing hand therapeutic exercises. Those that use the putty with less stress, tension or discomfort can achieve the rehabilitation goal of good health sooner and with less pain or inflammation.

These and other objects, features and advantages of the present invention will be apparent from the following detailed description of illustrative embodiments thereof.

### DETAILED DESCRIPTION OF THE PREFERRED EMBODIMENTS

The invention is directed to a therapeutic or exercise putty, in which CBD (cannabidiol) or CBG (cannabigerol) oil or powder and/or DMSO (dimethysulfoxide) is mixed with the base putty. The inclusion of these ingredients alone or together into the putty will help to reduce pain and inflammation of the treated hand during the putty use and permit proper safe rehabilitation and exercise by users and patients. The CBD or CBG oil or powder and DMSO ingredients inherently have properties to relieve pain and reduce inflammation. The CBD oil or powder and DMSO characteristics reveal upsides when used in topical applied products. There are many studies that have examined CBD oil and DMSO's role in easing chronic pain and inflammation. Findings have supported pain and inflammation reduction. Gels and creams with CBD oil or DMSO studied showed reduced joint inflammation and lesser signs of arthritic pain when used. This invention permits safe use during an exercise treatment or rehabilitation activity and can be used without dependence on narcotic or other orally administered pain killers.

The therapeutic base putty is much like the Dow Corning Q2-3233, which is boronated silicone elastomer. This elastomer may vary in viscosity and color. Generally, the Dow Corning Q2-3233 silicone elastomer will permit CBD or CBG oil or powder and/or DMSO to be mixed into the formula without consequence to the rehab exercise formula.

The CBD oil or powder ingredient stands for cannabidiol, and the CBG oil or powder stands for cannabigerol, each being an active ingredient in marijuana. However, this is not the marijuana component THC (tetrahydrocannabinol), which is associated with getting “high”. The CBD or CBG oil or powder ingredient is offered in a variety of products including some that are taken orally. Not all of the oral products have been proven safe or effective. Topical use, as intended in the rehab exercise putty of the present invention, can be done safely and the CBD or CBG oil or powder benefits can be realized. Inclusion of the CBD or CBG oil or powder ingredient disclosed herein, when part of the putty mixture, helps in the reduction of pain and inflammation. There are studies and claims among many topical and oral CBD oil products available that also reveal stress free, relaxation and less tense side effects that further contributes to the benefits associated with the intended rehabilitation and hand exercise.

The DMSO ingredient stands for Dimethyl Sulfoxide. DMSO is offered in a variety of products, including both high and lower concentrated topical creams or gels. It is used most frequently for pain relief in muscles and joints. When applied to the skin, DMSO is an excellent anti-inflammatory while relieving pain.

Use of DMSO in the putty formula of the present invention can transfer quickly to the skin during the exercise treatment. During the hand flexing motion and continuous gripping of the putty, the DMSO is expected to be realized and accelerated in a relatively brief period of time, such as about fifteen (15) minutes or less. The pain associated with the hand malady or the hand exercise when the therapeutic putty of the present invention is used can be reduced at the source, help to sustain prescribed rehabilitation and function to reduce inflammation.

In accordance with the present invention, when the putty mixture is with CBD or CBG oil or powder or DMSO or in combination together, the consequence of use is the greater likelihood of reduced pain and inflammation resulting in improved user health. This benefit is accomplished by creating a numbing feeling or anesthetic type reaction effect in the hand skin receptors, which means the signaling between the pain receptor nerves in the hand and the brain becomes normalized and sensitivity to pain is reduced. Relief from pain when there is less inflammation, hand swelling or stiffness is realized during the hand exercise and rehabilitation treatment when the therapeutic putty of the present invention is used.

The preferred formulations for such a therapeutic exercise putty, containing the CBD or CBG oil or powder or DMSO ingredients, together or each alone in the putty formula, disclosed herein or equivalents thereof, can be the Dow Corning Q2-3233 or an equivalent boronated silicone elastomer base putty mixed with CBD or CBG oil or powder in a preferred amount of the mixture from about 0.1% to about 10% of the total formula, or mixed with DMSO in a preferred amount of the mixture from about 0.1% to about 10% of the total formula, or mixed with a combination of CBD or CBG oil or powder and DMSO in a preferred amount of mixture from about 0.1% to about 10% of the total formula.

More specifically, the preferred prophetic formulations for the therapeutic putty of the present invention are as follows:
About 0.1% (percentage of total weight) CBD or CBG oil or powder or another ingredient which reduces pain and/or inflammation, plus about 99.9% (percentage of total weight) boronated silicone elastomer; to about 10% (percentage of total weight) CBD or CBG oil or powder or another ingredient which reduces pain and/or inflammation, plus about 90% (percentage of total weight) boronated silicone elastomer; or
About 0.1% (percentage of total weight) DMSO or another ingredient which reduces pain and/or inflammation, plus about 99.9% (percentage of total weight) boronated silicone elastomer; to about 10% (percentage of total weight) DMSO or another ingredient which reduces pain and/or inflammation, plus about 90% (percentage of total weight) boronated silicone elastomer; or
About 0.1% (percentage of total weight) combined CBD or CBG oil or powder and DMSO, or another ingredient which reduces pain and/or inflammation, plus about 99.9% (percentage of total weight) boronated silicone elastomer; to about 10% (percentage of total weight) combined CBD or CBG oil or powder and DMSO or another ingredient which reduces pain and/or inflammation, plus about 90% (percentage of total weight) boronated silicone elastomer.

Other secondary ingredients (dyes, analgesics, anti-microbials, etc.) may be added to the therapeutic putty that may reduce the approximate percentages in total weight of the boronated silicone elastomer in the prophetic formulations mentioned above, as long as the percentage of the CBD or CBG oil or powder ingredient or DMSO ingredient, when each is used without the other in the base putty, or the combination of the CBD or CBG oil or powder and DMSO ingredients together being used in the base putty, or another ingredient which reduces pain and/or inflammation, is in the putty in the range of about 0.1% of the total weight of the therapeutic putty product of the present invention and about 10% of the total weight of the therapeutic putty product of the present invention.

The therapeutic exercise putty of the present invention, such as disclosed in the above prophetic formulations, will aid in reducing pain and inflammation for a user or patient when human contact to the hand skin begins or is sustained during rehabilitation exercise or after the exercise ends. The putty mixture formulation of the present invention is used by a user or patient, thereby permitting longer exercise in order to have more effective rehabilitation with less suffering or illness. This pain reduction can be realized without the need for orally administered pain relief medications.

Although illustrative embodiments of the present invention have been described herein, it is to be understood that the invention is not limited to those precise embodiments, and that various other changes and modifications may be affected therein by one skilled in the art without departing from the scope or spirit of the invention.

What is claimed is:

1. A therapeutic or exercise putty, which comprises:
a silicone putty base; and
one or both of 1) a CBD (cannabidiol) or CBG (cannabigerol) oil or powder ingredient and 2) a DMSO (dimethysulfoxide) ingredient.

2. A therapeutic or exercise putty as defined by claim 1, wherein the silicone putty base is formed from boronated silicone elastomer.

3. A therapeutic or exercise putty as defined by claim 2, wherein the silicone putty base is Part No. Q2-3233 manufactured by The Dow Chemical Company of Midland, Michigan.

4. A therapeutic or exercise putty as defined by claim 2, wherein the CBD (cannabidiol) or CBG (cannabigerol) oil or powder ingredient is in the range of about 0.1% (percentage of total weight) to about 10% (percentage of total weight) when mixed with the silicone putty base without the DMSO (dimethysulfoxide) ingredient being added to the silicone putty base; and
wherein the boronated silicone elastomer is in the range of about 99.9% (percentage of total weight) to about 90% (percentage of total weight).

5. A therapeutic or exercise putty as defined by claim 2, wherein the DMSO (dimethysulfoxide) ingredient is in the range of about 0.1% (percentage of total weight) to about 10% (percentage of total weight) when mixed with the silicone putty base without the CBD (cannabidiol) or CBG (cannabigerol) oil or powder ingredient being added to the silicone putty base; and
wherein the boronated silicone elastomer is in the range of about 99.9% (percentage of total weight) to about 90% (percentage of total weight).

6. A therapeutic or exercise putty as defined by claim 2,
wherein both the CBD (cannabidiol) or CBG (cannabigerol) oil or powder ingredient and the DMSO (dimethysulfoxide) ingredient are mixed with the silicone putty base;
wherein the CBD (cannabidiol) or CBG (cannabigerol) oil or powder ingredient and the DMSO (dimethysulfoxide) ingredient together as a combined weight is in the range of about 0.1% (percentage of total weight) to about 10% (percentage of total weight); and
wherein the boronated silicone elastomer is in the range of about 99.9% (percentage of total weight) to about 90% (percentage of total weight).
